(19) United States
(12) Patent Application Publication
(10) Pub. No.: US 2012/0065634 A1
(43) Pub. Date: Mar. 15, 2012

(54) METHOD OF TREATING AN INTER-VERTEBRAL DISC

(75) Inventors: Sang-Heon LEE, Seoul (KR); Sung-Youn Cho, Uijeongbu-si (KR); Bong-Su Kang, Dongducheon-si (KR); Sang Wun Kim, Uijeongbu-si (KR); Ja-Kyo Koo, Seoul (KR); Jong-Tack Kim, Jeonju-si (KR)

(73) Assignees: KOREA UNIVERSITY INDUSTRIAL & ACADEMIC COLLABORATION FOUNDATION, Seoul (KR); U&I CORPORATION, Uijeongbu-si (KR)

(21) Appl. No.: 12/881,659

(22) Filed: Sep. 14, 2010

(51) Int. Cl. A61B 18/18

(52) U.S. Cl. 606/41

(57) ABSTRACT

The present invention relates to treating an inter-vertebral disc, in particular, to a method of treating an inter-vertebral disc by effectively ablating a tissue within a disc, in which herniation of nucleus pulposus occurs.

A method of treating an inter-vertebral disc according to one embodiment of the present invention relates to treatment using a guidable electrode to be inserted into an inter-vertebral disc of a patient, in which the treatment comprises a step of inserting a front end of a needle unit from the outside of annulus fibrosus into the inside of the annulus fibrosus, a step of inserting a guidable electrode through the rear of the needle unit, a step of adjusting position of the end of the electrode to be adjacent to a target tissue within the annulus fibrosus, and a step of ablating a tissue within nucleus pulposus by applying electrical energy to the electrode.

### BACKGROUND OF THE INVENTION

1. Field of the Invention

The present invention relates to treating an inter-vertebral disc, in particular, to a method of treating an inter-vertebral disc by effectively ablating a tissue within a disc, in which herniation of nucleus pulposus occurs.

2. Description of Prior Art

In general, when herniation of an inter-vertebral disc or herniation of nucleus pulposus (HNP) occurs, an inter-vertebral disc within the vertebra is extruded thereby compressing adjacent nerves and causing pains in the back and the lower part of the body. The inter-vertebral disc consists of nucleus pulposus and annulus fibrosus surrounding the nucleus pulposus. If the inner wall of the annulus fibrosus is torn by physical impact, the nucleus pulposus are spilled out through the torn inner wall of the annulus fibrosus due to pressure generated by weight upon standing up for a long time period or external impact.

In addition, the high pressure within the inter-vertebral disc is delivered to the outer surface of the inter-vertebral disc so that when part of the inter-vertebral disc is extruded, it develops into herniation of the inter-vertebral disc. The extruded inter-vertebral disc is not restored while continuing to maintain the extruded form. The extruded portion compresses nerves around the vertebra thereby causing a back pain (FIG. 1).

With regard to a method for treating herniation of an inter-vertebral disc, there is a restoring method or reduced pressure treatment, which ablates component materials of nucleus pulposus within an inter-vertebral disc to reduce pressure in the inside of the inter-vertebral disc so that the extruded portion of the inter-vertebral disc is spontaneously restored to the inside of the inter-vertebral disc.

In general, with regard to reduced pressure treatment, there is a method of inserting a radio frequency electrode into a tissue within an inter-vertebral disc and then applying radio frequency to ablate a tissue around the electrode in the gas state that component materials of nucleus pulposus are divided into electrons having negative charges and ions having positive charges by an electric field, with high energy, formed by using the radio frequency.

The method of ablating a tissue in the body by using a radio frequency electrode is advantageous in that it can reduce hospitalization and significantly reduce operation costs, and reduce the risk of side effects even after operation, compared to surgical operation.

Radio frequency has a frequency range of 100 kHz to 20,000 kHz. U.S. Pat. No. 6,554,827 and others disclose a method of ablating or removing a tissue of a human body and removing wastes and others in blood vessels by using a radio frequency electrode.

The method using a radio frequency electrode ablates a nucleus pulposus tissue within an inter-vertebral disc to reduce internal pressure of the inter-vertebral disc thereby restoring the herniated inter-vertebral disc portion. Thus, as a tissue in proximity to the herniated portion is directly ablated, the treatment effect increases. Ablating a tissue distant from the herniated portion cannot achieve a sufficiently desired effect.

A conventional radio frequency electrode is generally in a straight form, which reaches only into a portion positioned in a straight line from a position in the body into which the electrode is inserted. Thus, portions into which the electrode reaches are limited. In particular, when the electrode is used to treat herniation of an inter-vertebral disc, due to the existence of the vertebra and nerves around the vertebra, positions into which the radio frequency electrode can be inserted are very limited. Thus, it becomes difficult to ablate a tissue opposite to a position into which the radio frequency electrode is inserted.

Accordingly, there have been suggested treatment means to increase a treatment effect by applying a guidable electrode, in which one end to be inserted is configured with a flexible body, and ablating an internal tissue at a location in extremely close proximity to the target tissue to be ablated.

In addition, if one end is configured with a flexible body, it is preferable to make a diameter of the end of the electrode smaller than that of a conventional electrode. In that case, the electrode can easily reach into a target portion through an internal radius of a needle which is inserted into the body.

However, the method of treating inter-vertebral disorders such as herniation of nucleus pulposus by using an electrode configured with a flexible body and having a small diameter as described above is different from a conventional method using “a rigid electrode which is not guidable” with respect to a method of reaching into a target tissue and treatment processes.

Furthermore, since a herniated nucleus pulposus portion is adjacent to vertebral nerves, in order to provide electrical surgical treatment, which is applied directly thereto and achieves a safer and effective treatment effect, a new method for treatment of an inter-vertebral disc using a guidable electrode has been demanded.

### SUMMARY OF THE INVENTION

The present invention has been made to resolve the foregoing problems, and its object is to provide a safe treatment method of an inter-vertebral disc, which effectively reaches into a herniated disc portion to increase a treatment effect and not damage sensitive portions such as vertebral nervous tissues.

In order to achieve the object, a method of treating an inter-vertebral disc according to one embodiment of the present invention relates to treatment using a guidable electrode to be inserted into an inter-vertebral disc of a patient, in which the treatment comprises a step of inserting a front end of a needle unit from the outside of annulus fibrosus into the inside of the annulus fibrosus, a step of inserting the guidable electrode through the rear of the needle unit, a step of adjusting position of the end of the electrode to be adjacent to a target tissue within the annulus fibrosus, and a step of ablating a tissue within nucleus pulposus by applying electrical energy to the electrode.

The step of ablating a tissue within nucleus pulposus by applying electrical energy to the electrode is characterized in that in order to increase contact between the target tissue and the electrode in the state that the end of the electrode is bent, the electrode rotates based on an axis of the electrode and moves in the direction being spaced from the target tissue thereby ablating the tissue.

In addition, the step of adjusting position of the end of the electrode to be adjacent to a target tissue within the annulus fibrosus comprises the following steps: firstly, positioning the end of the electrode in proximity to a target tissue within the annulus fibrosus and applying electrical energy; secondly, identifying whether patient's nerves respond in the first step; thirdly, if patent's nerves respond, moving the end of the electrode in the direction being spaced from the target tissue and then applying electrical energy again to identify whether patient's nerves respond, and fourthly, if patient's nerves do not respond, determining the position of the end of the electrode. The object of the present invention described above can be achieved by the foregoing characteristics.

Furthermore, a method of treating an inter-vertebral disc according to another embodiment of the present invention relates to treatment using a guidable electrode to be inserted into an inter-vertebral disc of a patient, in which the treatment comprises a step of inserting a front end of a needle unit through annulus fibrosus to be positioned at an extruded disc portion, a step of inserting a guidable electrode through the rear of the needle unit, a step of adjusting position of the end of the electrode to be adjacent to a target tissue of the extruded disc portion, and a step of ablating the tissue within the extruded disc portion by applying electrical energy to the electrode.

### BRIEF DESCRIPTION OF DRAWINGS

FIG. 1 is a cross-sectional view of the vertebra for explanation of the symptom that part of an inter-vertebral disc is herniated thereby compressing vertebral nerves.

FIG. 2 is a view for explanation of treatment processes using a rigid needle in a disc treatment method according to one embodiment of the present invention.

FIG. 3 is a view for explanation of treatment processes using a flexible needle according to one embodiment of the present invention.

FIG. 4 is a view for explanation of movement of an electrode in a disc treatment method according to one embodiment of the present invention.

FIG. 5 is a view for explanation of a step of setting position of an electrode during treatment processes according to a disc treatment method in one embodiment of the present invention.

FIG. 6 is a tomography view for explanation of treatment processes according to a disc treatment method in one embodiment of the present invention.

FIG. 7 is a view for explanation of treatment processes according to a disc treatment method in another embodiment of the present invention.

FIG. 8 is a view for explanation of treatment processes according to a disc treatment method in another embodiment of the present invention.

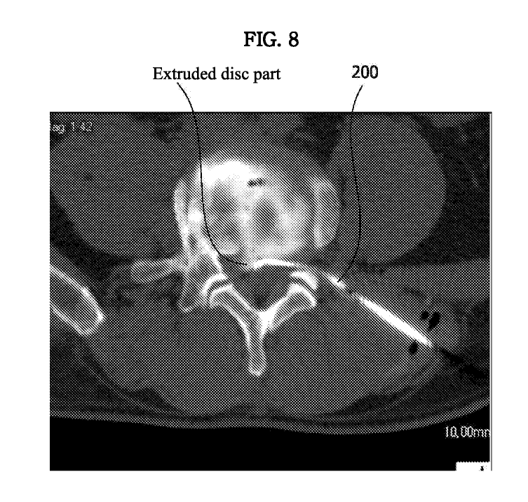

### DETAILED DESCRIPTION OF THE PREFERRED EMBODIMENTS

Hereinafter, a method of treating an inter-vertebral disc according to one embodiment of the present invention will be described in detail with reference to the accompanying drawings.

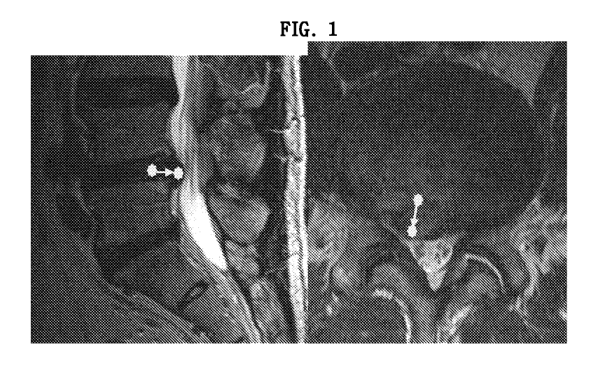

With reference to FIG. 1, a method of treating an inter-vertebral disc according to one embodiment of the present invention relates to treatment using a guidable electrode to be inserted into an inter-vertebral disc of a patient, in which the treatment comprises a step of inserting a front end of a needle unit from the outside of annulus fibrosus into the inside of the annulus fibrosus, a step of inserting the guidable electrode through the rear of the needle unit, a step of adjusting position of the end of the electrode to be adjacent to a target tissue within the annulus fibrosus, and a step of ablating a tissue within nucleus pulposus by applying electrical energy to the electrode.

The needle unit may be configured by various means, such as a metal or synthetic resin needle, which act as a passage from the outside into the inside of a disc. The needle unit is inserted at the back portion of a patient and passes through annulus fibrosus of a disc to be treated such that the front end of the needle unit is positioned at a portion to be treated.

Accordingly, once the needle unit is inserted so as to form a passage toward a portion to be treated in a patient, a guidable electrode is inserted through the rear of the needle unit so that treatment of a target tissue is possible.

In addition, the needle unit may be configured by a needle made of rigid material, a flexible needle coupled into an internal radius of the needle, and a trocar coupled into an internal radius of the flexible needle. The step of inserting a guidable electrode through the rear of the needle unit may comprise a step of removing the trocar when the front end of the needle unit is positioned within annulus fibrosus, and a step of inserting a guidable electrode through the rear of the flexible needle.

The needle (100) generally has a tube shape in the form of a shaft made of metal material and acts as a passage, which is inserted into a portion of the body to be directly treated.

In addition, the needle (100) is configured to have a front end to be inserted, which has a diagonal cross section, like a syringe needle. Thus, it can be easily inserted into the body without the need of separate equipment. However, since the needle (100) is inserted into the body to perform as a guiding tube during treatment, it generally has the shape of a pipe cross section, in which the end is not sharp for safety.

The flexible needle (200) is detachably coupled into the internal radius of the needle and made of a synthetic resin, which is relatively soft material. The flexible needle (200) is preferably longer than the needle (100) so as to be projected from the front end of the needle (100).

Accordingly, once the flexible needle (200) is coupled into the needle (100), a treatment instrument to be inserted into the internal radius of the flexible needle (200) can be guided into the body while contacting directly with the soft flexible needle (200) without contacting with the needle (100) made of metal material.

In addition, a trocar (300), called a hollow needle, is coupled into the internal radius of the flexible needle (200) and projected from the front end of the flexible needle (200) so that it can be easily inserted into the body and constitutes the needle unit as a whole.

The front end of the needle unit, from which the trocar (300) is projected, is inserted into the body. The flexible needle (200) is coupled into the needle (100), and the trocar (300) is coupled into the flexible needle (200). Thus, insertion into a body tissue with sufficient rigidity is possible.

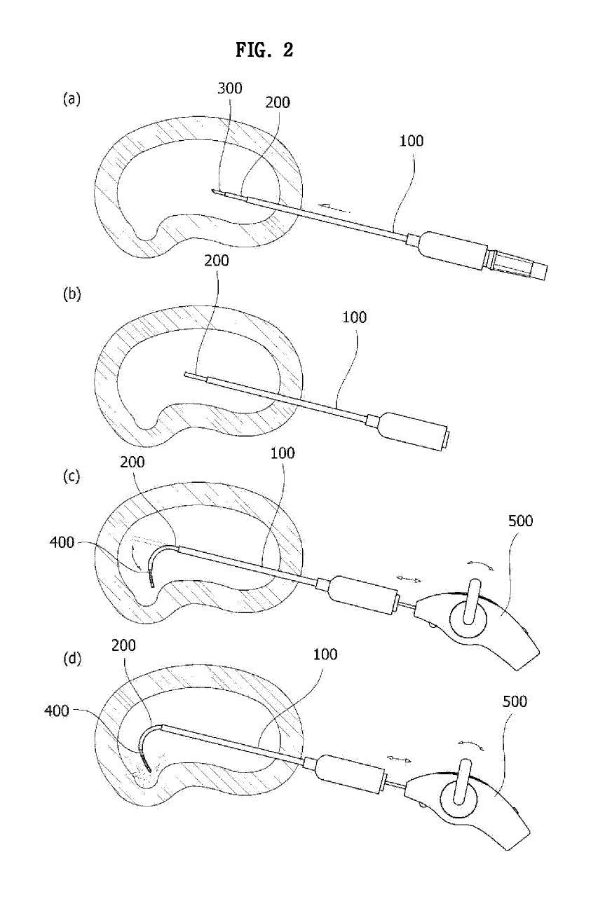

The needle unit is inserted into a body tissue. In general, a portion into which the needle unit is inserted for disc treatment is opposite to a herniated portion of a disc. The front end of the needle unit is inserted at the back skin, passes through annulus fibrosus of the disc, and advances until it is positioned within the disc (FIG. 2a).

When the front end of the needle unit is positioned within annulus fibrosus, the trocar (300) is removed so that a passage externally configured by the needle (100) and internally configured by the flexible needle (200) is formed (FIG. 2b).

In addition, a guidable electrode (400) is inserted through the rear of the flexible needle (200). Since the flexible needle (200) is coupled into the needle (100) to be projected from the end of the needle (100), and also is made of flexible material, the electrode is inserted such that the end of the electrode is projected from the end of the flexible needle (200), and the position of the end of the electrode can be adjusted such that the end of the electrode is guided to be adjacent to a target tissue within annulus fibrosus (FIG. 2c).

The effect in treatment using reduced pressure within a disc is the most effective if ablation of an internal tissue is performed at a location in proximity to an extruded portion as described above. Thus, it is ideal to perform treatment by positioning the end of the electrode (400) in extremely close proximity to an extruded portion as a target point (FIG. 2d).

When the electrode (400) is positioned at the target point, electrical energy is applied to the electrode (400) to ablate a tissue within nucleus pulposus. The pressure in the inside of the disc is lowered by the volume of the ablated tissue so that the disc portion extruded toward the outside is restored. As a result, compression in vertebral nerves is reduced thereby relieving the back pain.

Furthermore, the needle unit may be configured by a flexible needle and a trocar coupled into an internal radius of the flexible needle. The step of inserting a guidable electrode through the rear of the needle unit may comprise a step of removing the trocar when the front end of the needle unit is positioned within annulus fibrosus, and a step of inserting a guidable electrode through the rear of the flexible needle.

Treatment using this needle unit is performed with processes similar to the rocesses that have been described.

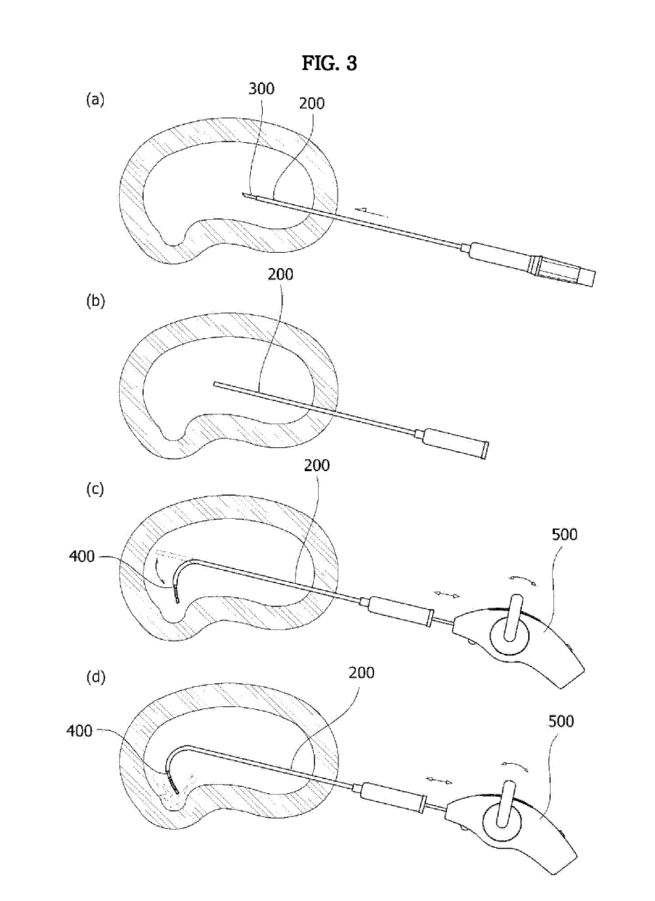

With reference to FIG. 3, the needle unit is inserted into a body tissue (FIG. 3a). When the front end of the needle unit is positioned within annulus fibrosus, the trocar (300) is removed so that a passage from the outside into the inside through the inside of the flexible needle (200) is formed. As a whole, the flexible needle (200) alone acts as a passage passing though the annulus fibrosus and extending into the inside of the disc (FIG. 3b).

When the trocar (300) is removed, the flexible needle (200) may be taken out together. Thus, it should be noted that the needle be removed while tightly holding the flexible needle (200) by hand and maintaining the position.

Likewise, the guidable electrode (400) is inserted through the rear of the flexible needle (200). Since it is likely that the flexible needle (200) is curved or bent, it is desirable to slowly and smoothly insert the electrode into the flexible needle (200) (FIG. 3c).

In addition, the end of the electrode (400) is guidable, and the flexible needle (200) also is made of flexible material. Thus, the electrode (400) and the flexible needle (200) are bent together so that position of the end of the electrode can be adjusted to be adjacent to a target tissue within annulus fibrosus (FIG. 3c), and treatment can be performed by positioning the end of the electrode (400) in extremely close proximity to a target point (FIG. 3d).

Unlike when ablating central nucleus pulposus of a disc, when ablating nucleus pulposus of an extruded portion, sensitive portions such as adjacent vertebral nervous tissues may exist. Thus, it is safe to identify whether nervous tissues exist prior to determining the position of the electrode and ablate the nucleus pulposus by taking out the electrode in the direction being spaced from the final position of the electrode.

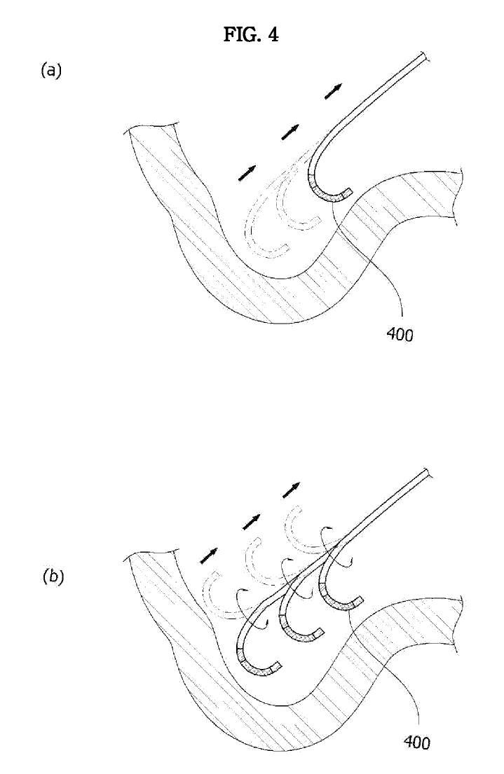

Accordingly, the step of ablating a tissue within nucleus pulposus by applying electrical energy to the electrode is characterized in that the electrode moves in the direction being spaced from a target tissue thereby ablating the tissue (FIG. 4a).

In addition, as many tissues are contacted with the electrode, many tissues are ablated. Thus, in order to increase contact between target tissues and the electrode in the state that the end of the electrode is bent, the electrode may rotate based on an axis of the electrode thereby ablating tissues.

Furthermore, when ablating a tissue within nucleus pulposus by applying electrical energy to the electrode, in order to increase contact between the target tissue and the electrode in the state that the end of the electrode is bent, the electrode may rotate based on an axis of the electrode and move in the direction being spaced from the target tissue thereby ablating the tissue (FIG. 4b).

As described, since there are many sensitive portions around a herniated portion of a disc, when ablating a tissue within the herniated portion of the disc, adjacent nervous tissues may be damaged thereby causing side effects.

Thus, the surgeon identifies whether nervous tissues exist around the electrode (400) prior to ablating a tissue within a disc and then performs tissue ablation treatment to perform safer treatment.

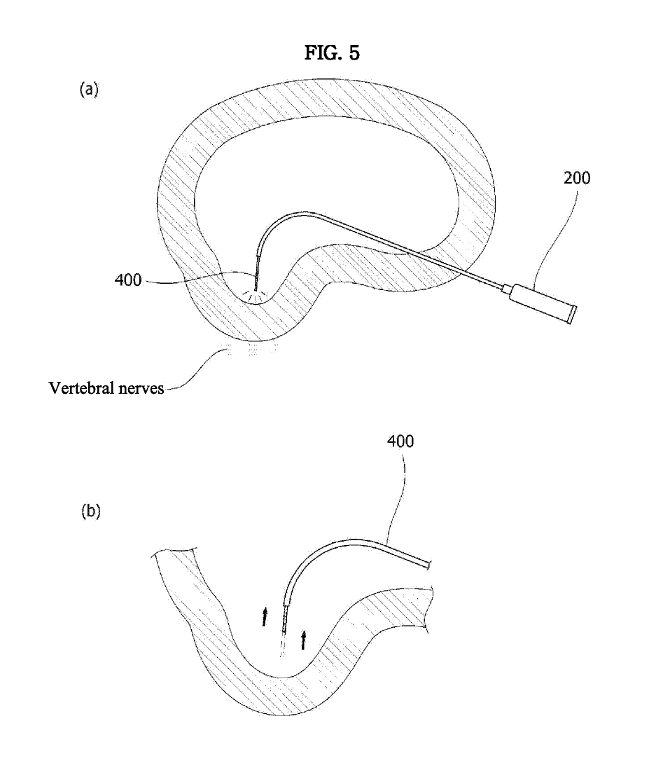

For example, in the step of adjusting position of the end of the electrode to be adjacent to a target tissue in annulus fibrosus, the end of the electrode is positioned in proximity to the target tissue in the annulus fibrosus, and electrical energy is applied to identify whether patient's nerves respond in the step. If patient's nerves respond, tissue ablation is not performed (FIG. 5a).

Accordingly, the surgeon moves the end of the electrode in the direction being spaced from the target tissue, and then applies electrical energy again to identify whether nerves respond. If nerves do not respond, the surgeon determines the position of the end of the electrode and moves the electrode in the direction being spaced from the position thereby ablating the tissue (FIG. 5b).

In addition, in the step of ablating a tissue in nucleus pulposus by applying electrical energy to the electrode, time for applying electrical energy may be limited to less than five seconds to prevent temperature in a portion to be treated from being overly high.

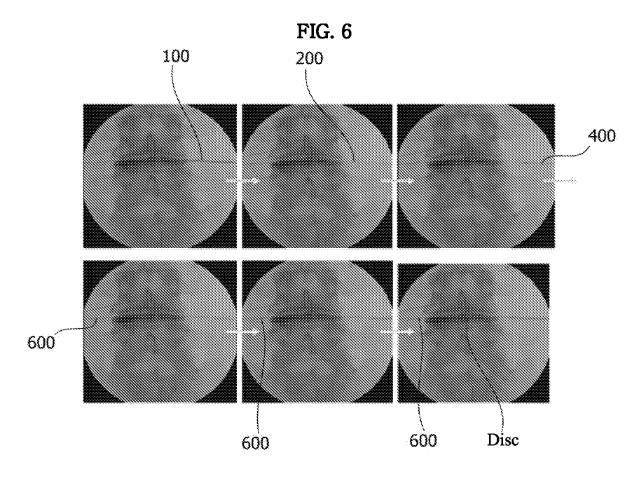

Furthermore, a separate fluid passage (600), other than a path for insertion of the electrode, is provided to further comprise a step of supplying cooling fluid to cool a portion to be treated so that safer and less painful treatment can be performed (FIG. 6).

In addition, a method of treating an inter-vertebral disc according to one embodiment of the present invention uses radio frequency and can be applied to various cases, including herniation of an inter-vertebral disc and even the case where reaching into a target tissue with an electrode in a straight form is difficult, to perform local ablation. An electrode suitable for minimal invasive surgery (MIS) can be realized.

Accordingly, since the state of internal tissues after ablation of nucleus pulposus within a disc becomes fine, the present invention may further comprise a step of inserting an implant, which is an artificial structure swelling within a disc such as a spinal cage, through the guiding tube. Compared to a method using a conventional electrode, effective treatment, which has good prognosis, can be performed.

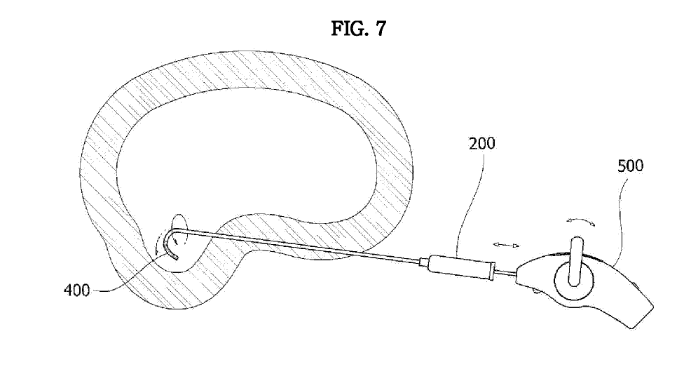

With respect to FIG. 7, a method of treating an inter-vertebral disc according to another embodiment of the present invention relates to treatment using a guidable electrode to be inserted into an inter-vertebral disc of a patient, in which the treatment comprises a step of inserting a front end of a needle unit through annulus fibrosus to be positioned at an extruded disc portion, a step of inserting a guidable electrode through the rear of the needle unit, a step of adjusting position of the end of the electrode to be adjacent to a target tissue of the extruded disc portion, and a step of ablating the tissue within the extruded disc portion by applying electrical energy to the electrode.

The method of treating an inter-vertebral disc according to another embodiment is a method which, if herniation of a disc is severe, directly inserts the needle unit into the extruded portion of the disc and positions the front end of the electrode therein to ablate a tissue.

Accordingly, the front end of the needle unit is inserted directly into an extruded disc portion outside annulus fibrosus without passing through the inside of the annulus fibrosus. As described in the embodiments that have been described, electrical energy is applied to the electrode thereby ablating a nucleus pulposus tissue within the extruded disc.

The needle unit is configured by a needle made of rigid material, a flexible needle coupled into an internal radius of the needle, and a trocar coupled into an internal radius of the flexible needle. The step of inserting a guidable electrode through the rear of the needle unit may comprise a step of removing the trocar when the front end of the needle unit is positioned at the extruded disc portion, and a step of inserting a guidable electrode through the rear of the flexible needle.

In addition, the needle unit is configured by a flexible needle and a trocar coupled into an internal radius of the flexible needle. The step of inserting a guidable electrode through the rear of the needle unit may comprise a step of removing the trocar when the front end of the needle unit is positioned at the extruded disc portion, and the step of inserting a guidable electrode through the rear of the flexible needle.

The treatment method according to the above configuration example is the same as a method of treating an inter-vertebral disc according to one embodiment of the present invention.

Furthermore, as described, a tissue may be ablated by moving the electrode in the direction being spaced from the target tissue or rotating the electrode based on an axis of the electrode to increase contact between the target tissue and the electrode in the state that the end of the electrode is bent. Performing these processes together is possible (FIG. 7).

In addition, the step of positioning the end of the electrode at a tissue within an extruded disc may comprise the following steps: firstly, positioning the end of the electrode in proximity to a target tissue of an extruded disc portion and applying electrical energy; secondly, identifying whether patient's nerves respond in the first step; thirdly, if patient's nerves respond, moving the end of the electrode in the direction being spaced from the target tissue and then applying electrical energy again to identify whether nerves respond; and fourthly, if nerves do not respond, determining the position of the end of the electrode. This treatment also is identical to the treatment method that has been described and can prevent damage to important tissues.

The present invention is not limited to the embodiments that have been described and can be modified by one of ordinary skill in the art within the scope of the technical idea of the present invention. Accordingly, the scope of protection sought by the present invention is not limited to the descriptions in the specification and should be defined by the claims and the technical idea defined therein.

1. A method of treating an inter-vertebral disc using a guidable electrode to be inserted into an inter-vertebral disc of a patient, comprising
a step of inserting a front end of a needle unit from the outside of annulus fibrosus into the inside of the annulus fibrosus,
a step of adjusting position of the end of the electrode to be adjacent to a target tissue within the annulus fibrosus; and
a step of ablating a tissue in nucleus pulposus by applying electrical energy to the electrode.

2. The method of treating an inter-vertebral disc claimed in claim 1, wherein the needle unit is configured by a needle made of rigid material, a flexible needle coupled into an internal radius of the needle, and a trocar coupled into an internal radius of the flexible needle, and
the step of inserting a guidable electrode through the rear of the needle unit comprises a step of removing the trocar when the front end of the needle unit is positioned within the annulus fibrosus, and a step of inserting a guidable electrode through the rear of the flexible needle.

3. The method of treating an inter-vertebral disc claimed in claim 1, wherein the needle unit is configured by a flexible needle and a trocar coupled into an internal radius of the flexible unit, and
the step of inserting a guidable electrode through the rear of the needle unit comprises a step of removing the trocar when the front end of the needle unit is positioned within the annulus fibrosus, and a step of inserting a guidable electrode through the rear of the flexible needle.

4. The method of treating an inter-vertebral disc claimed in claim 1, wherein the step of ablating a tissue within nucleus pulposus by applying electrical energy to the electrode is characterized in that the electrode moves in the direction being spaced from a target tissue thereby ablating the tissue.

5. The method of treating an inter-vertebral disc claimed in claim 1, wherein the step of ablating a tissue within nucleus pulposus by applying electrical energy to the electrode is characterized in that in order to increase contact between a target tissue and the electrode in the state that the end of the electrode is bent, the electrode rotates based on an axis of the electrode thereby ablating the tissue.

6. The method of treating an inter-vertebral disc claimed in claim 1, wherein the step of ablating a tissue within nucleus pulposus by applying electrical energy to the electrode is characterized in that in order to increase contact between a target tissue and the electrode in the state that the end of the electrode is bent, the electrode rotates based on an axis of the electrode and moves in the direction being spaced from the target tissue thereby ablating the tissue.

7. The method of treating an inter-vertebral disc claimed in claim 1, wherein other than a path for insertion of the electrode, a separate fluid passage, which reaches into a target tissue, is provided to further comprise a step of supplying cooling fluid.

8. The method of treating an inter-vertebral disc claimed in claim 1, wherein the step of adjusting position of the end of the electrode to be adjacent to a target tissue in annulus fibrosus comprises the following steps:
firstly, positioning the end of the electrode in proximity to a target tissue within the annulus fibrosus and applying electrical energy,
secondly, identifying whether patient's nerves respond in the first step,
thirdly, if patient's nerves respond, moving the end of the electrode in the direction being spaced from the target tissue, and then applying electrical energy again to identify whether nerves respond, and
fourthly, if nerves do not respond, determining the position of the end of the electrode.

9. The method of treating an inter-vertebral disc claimed in claim 8, wherein the method further comprises a step of inserting an artificial implant into a disc through a guiding tube after ablating a tissue within nucleus pulposus by applying electrical energy to the electrode.

10. A method of treating an inter-vertebral disc using a guidable electrode to be inserted into an inter-vertebral disc of a patient, comprising
a step of inserting a front end of a needle unit through annulus fibrosus to be positioned at an extruded disc portion,
a step of inserting a guidable electrode through the rear of the needle unit,
a step of adjusting position of the end of the electrode to be adjacent to a target tissue of an extruded disc portion, and
a step of ablating the tissue within the extruded disc portion by applying electrical energy to the electrode.

11. The method of treating an inter-vertebral disc claimed in claim 10, wherein the needle unit is configured by a needle made of rigid material, a flexible needle coupled into an internal radius of the needle, and a trocar coupled into an internal radius of the flexible needle, and
the step of inserting a guidable electrode through the rear of the needle unit comprises a step of removing the trocar when the front end of the needle unit is positioned at an extruded disc portion, and a step of inserting a guidable electrode through the rear of the flexible needle.

12. The method of treating an inter-vertebral disc claimed in claim 10, wherein the needle unit is configured by a flexible needle, and a trocar coupled into an internal radius of the flexible needle, and
the step of inserting a guidable electrode through the rear of the needle unit comprises a step of removing the trocar when the front end of the needle unit is positioned at an extruded disc portion, and a step of inserting a guidable electrode through the rear of the flexible needle.

13. The method of treating an inter-vertebral disc claimed in claim 10, wherein the step of ablating a tissue within nucleus pulposus by applying electrical energy to the electrode is characterized in that in order to increase contact between the target tissue and the electrode in the state that the end of the electrode is bent, the electrode rotates based on an axis of the electrode thereby ablating the tissue.

14. The method of treating an inter-vertebral disc claimed in claim 10, wherein the step of positioning the end of the electrode at a tissue within an extruded disc portion comprises the following steps:
firstly, positioning the end of the electrode in proximity to a target tissue of an extruded disc portion and applying electrical energy,
secondly, identifying whether patient's nerves respond in the first step,
thirdly, if patient's nerves respond, moving the end of the electrode in the direction being spaced from the target tissue and then applying electrical energy again to identify whether nerves respond, and
fourthly, if nerves do not respond, determining the position of the end of the electrode.
